(19) United States
(12) Patent Application Publication
(10) Pub. No.: US 2017/0105877 A1
(43) Pub. Date: Apr. 20, 2017

(54) WOUND DRESSING

(71) Applicant: M4 Medical Pty Ltd, Darwin (AU)

(72) Inventors: Jeffrey Buteux, Darwin (AU); Debra Lee Bisogno, Darwin (AU); Matthew Paul Pullin, Darwin (AU)

(21) Appl. No.: 15/317,046

(22) PCT Filed: May 18, 2015
(86) PCT No.: PCT/AU2015/050250

§ 371 Date: Dec. 7, 2016

(30) Foreign Application Priority Data
Jun. 10, 2014 (CN) 201410254250.8
Jun. 10, 2014 (CN) 201420304610.6

(51) Int. Cl. A61F 13/00; A61F 13/10; A61F 13/06; A61F 13/12; A61F 13/14; A61L 15/40; B65D 75/36; B65D 83/08; B65D 75/52; B65B 55/12; A61L 15/42; A61L 15/44; A61F 7/10; A61F 15/00

(52) U.S. Cl. CPC A61F 13/00021; A61F 13/00063; A61F 7/106; A61F 13/105; A61F 13/068; A61F 13/12; A61F 13/14; A61F 13/00038; A61F 13/00076; A61F 15/002; A61F 13/00987; B65D 75/366; B65D 75/367; B65D 83/0841; B65D 75/52; B65B 55/12; A61L 15/42; A61L 15/44; A61L 15/40; A61F 2007/108; A61F 2013/0028; A61F 2013/00119; A61F 2013/00153

(57) ABSTRACT

Some embodiments relate to a wound dressing, packages containing such dressings and methods of their manufacture. Such wound dressings may comprise: a cohesive bandage having a length and a width, the length being substantially greater than the width; and an absorbent pad affixed to the bandage near to but spaced from a terminal end of the bandage so as to form a tail portion between the pad and the terminal end of the bandage.

### TECHNICAL FIELD

The present application relates generally to a wound dressing, for example such as a compression dressing.

### BACKGROUND

A wide variety of materials have been used to treat wounds using different types of dressings. Typical materials that are used in dressings include individual wound contact materials, absorbent materials, adhesive binding and non-adhesive binding materials and fastening materials. Dressings are also available that integrate these components and are manufactured as a single item. These range from small dressings such as adhesive band aid type and larger adhesive dressings. Dressings provide numerous conditions, which are beneficial to health including limitation of bleeding, providing aseptic covering, providing a beneficial compressive force/support and many others which promote healing or limit injury.

The Invention described in this application is designed to be used for treatment of a wide variety of wounds and be substituted for dressings for simple traumatic wounds and for more complex and larger wounds. The invention can also be used as a compression bandage for application to provide first aid for envenomation or crush injury, as a dressing for chronic wounds and as a support bandage for injury. Dressings such as band aid type bandages, combinations of wound contact materials, absorbent materials, sticking or non-sticking binding materials and fastening materials and single piece, multiple component dressings can be replaced by some embodiments described herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

Some embodiments relate to a wound dressing comprising: a cohesive bandage having a length and a width, the length being substantially greater than the width; and an absorbent pad affixed to the bandage near to but spaced from a terminal end of the bandage so as to form a tail portion between the pad and the terminal end of the bandage.

The dressing may be a compression dressing adapted to exert a compressive force to a body part when wrapped around the body part.

The length of the tail portion may be between about 10 mm and about 100 mm, optionally between about 20 mm and about 40 mm, optionally between about 25 mm and about 35 mm, optionally between about 60 mm and about 80 mm, optionally between about 65 mm and about 75 mm. The length of the tail portion may be between about 1% and 10% of the length of the bandage, optionally about 5-10%, optionally about 6-8%, optionally about 7%.

The width of the pad in a direction across the width of the bandage may between about 10 mm and about 30 mm, optionally between about 40 mm and about 60 mm, optionally between about 65 mm and about 85 mm, optionally between about 90 mm and about 110 mm, optionally between about 140 mm and about 160 mm. The width of the pad 340 may be between about 25% and 400% of the length of the pad 340, optionally about 50-300%, optionally about 60-200%, optionally about 80-120%, optionally about 100%, optionally about 40-100%, optionally about 50-60%. The width of the pad 340 may be between about 50% and 200% of the width of the bandage 320, optionally about 75-120%, optionally about 100%. The width of the pad may be approximately equal to the width of the bandage. The thickness of the pad 340 may be between about 0.5 mm and 7 mm, optionally about 2-4 mm, optionally about 2 mm, optionally about 2.5 mm, optionally about 3 mm, optionally about 4 mm.

The dressing may be sized to wrap around a human digit. The dressing may be sized to wrap a human limb, human head or human trunk such as a chest or abdomen. The dressing may be sized to wrap around a human torso.

In some embodiments, the pad may comprise at least one therapeutic additive. The at least one additive may be selected from the group of classes comprising: medication, drugs, ointments, antimicrobials, antibiotics, antiseptics, coagulants, hydrogels, analgesics, anaesthetics, anti-inflammatories and antihistamines. The at least one additive may be selected from the group comprising: silver derivatives, iodine derivatives, petroleum derivatives, saline, chitosan, hydrofibres, hydrogels, alginate, Melaleuca oil, antimicrobial oils and honey. The at least one additive may comprise a coagulant selected from the group comprising: cellulose-based agents, gelatin-based agents, collagen-based agents, fibrin-based agents, thrombin-based agents, chitin-based agents, chitosan-based agents and mineral-based agents. The at least one additive may comprise a hydrogel including Melaleuca oil.

In some embodiments, the dressing may further comprise a cold pack. The cold pack may be disposed between the bandage and the pad. The dressing may further comprise one of a sleeve, slot, pouch or pocket to receive the cold pack. In some embodiments, the cold pack can be activated to start an endothermic reaction to provide a cooling effect. The cold pack may comprise: an outer bag; a first substance disposed within the outer bag; an inner frangible bag disposed within the outer bag; and a second substance disposed within the inner bag; wherein, prior to activation, the first and second substances are isolated from each other by the inner bag, wherein the pack is activated by breaking the inner bag, and wherein, after the pack is activated, the substances are allowed to mix to start an endothermic reaction. The second substance may comprise water and the first substance may comprise one or more substances selected from the group comprising: ammonium nitrate, calcium ammonium nitrate and urea.

In some embodiments, the dressing may further comprise a non-adhesive tab affixed to the terminal end of the bandage, the tab being formed of a different material than a material of the bandage.

In some embodiments, the pad may comprise one or more foldable wings to extend a contact area of the pad.

Some embodiments relate to a compression dressing, comprising: an elastic bandage strip being designed for exerting a compressive force when wrapped around a body part sufficient to hold the compression dressing in place; and an absorbent pad being affixed to an inner side of the elastic bandage strip and close to a terminal end of the elastic bandage strip.

The elastic bandage strip may have self-adhesive properties and may be designed not to adhere to clothing, hair, skin or latex gloves. The elastic bandage strip may be constructed so as to have self-adhesive properties sufficient to cause two adjacent layers of the compression dressing to remain adhered to one another under elastic extension or pressure. The elastic bandage strip may be constructed of non-woven elastomeric fibres or of woven cotton type having firm elastic extendibility in longitudinal and vertical directions, with an adhesive attached thereto. The elastic bandage strip may have self-adhesive properties sufficient to cause two adjacent layers of the compression dressing to remain adhered to one another without the use of a fastening mechanism. The elastic bandage strip may be a cohesive bandage.

The absorbent pad may be attached to the elastic bandage strip by stitching, welding, needle tacking or bonding. The absorbent pad may be constructed of foamed, woven or nonwoven material of natural or synthetic fibres. The absorbent pad may have a width equal to or smaller than that of the elastic bandage strip. The distance between the absorbent pad and the terminal end of the elastic bandage strip may be between about 10 mm to 100 mm, optionally about 50 mm to 70 mm.

In some embodiments, the compression dressing may be loosely rolled into a roll and packaged in a sterile packaging.

In some embodiments, a non-adhesive covering may be arranged on the exposed surface of the absorbent pad.

Some embodiments relate to a wound dressing product comprising a package and one of the described dressings sealed within the package. At least part of the package may be formed of a material which allows the dressing to be sterilised within the package. The material may have a gas permeability high enough to allow gas sterilisation of the dressing within the package. At least part of the package may be formed of a transparent material to allow a user to see at least part of the dressing while the dressing remains sealed within the package.

The dressing may be in a rolled or folded configuration within the package. The dressing may be rolled with the pad disposed near an outer edge of the roll. A wound contact surface of the pad may face towards or away from the centre of the roll. The tail may be rolled or folded to cover the wound contact surface of the pad.

In some embodiments, the package may comprise a blister pack including one or more chambers, each chamber may define a recess and an opening, each recess may contain one of the dressings, and each opening may be sealed by a sheet.

In some embodiments, the package may comprise a dressing dispenser containing a roll of a plurality of the dressings connected end to end. The dressings may be manually separable from one another. The roll of dressings may further comprise indicia printed on the dressings to indicate where one dressing ends and the next adjacent dressing begins. The dispenser may further comprise an edge or blade for separating dressings from the roll. The roll of dressings may further comprise frangible or pre-weakened areas such as a perforated line between adjacent dressings to assist in separating the dressings from each other and the roll. The dispenser may further comprise a hub at the centre of the roll, the hub being pivotally mounted on a body of the dispenser to allow the roll to rotate relative to the body.

Some embodiments relate to a wound dressing kit comprising a plurality of the described wound dressing products. The kit may comprise a kit package to contain the plurality of wound dressing products. The plurality of products may include a range of different dressing sizes. The plurality of products may include a range of different dressing types. The kit may include more than one dressing of each size. The kit may include more than one dressing of each type.

Each package of the plurality of products may include indicia to indicate at least one of a size and a type of the dressing contained within. The plurality of products may be colour coded by at least one of a size and a type such that each dressing of the plurality of products is coloured to indicate a respective at least one of size and type of the dressing.

Some embodiments relate to a method of manufacturing one of the described wound dressing products. The method may comprise forming the dressing; enclosing the dressing in the package; and sealing the package. The method may further comprise sterilising the dressing in the package.

Some embodiments relate to a method of manufacturing a dressing comprising: forming a cohesive bandage having a length and a width, the length being substantially greater than the width; and affixing an absorbent pad to the bandage near to but spaced from a terminal end of the bandage so as to form a tail portion between the pad and the terminal end of the bandage.

The method may further comprise providing the pad with one or more therapeutic additives. The method may further comprise sealing the dressing in a package. The method may further comprise sterilising the dressing within the sealed package.

### BRIEF DESCRIPTION OF DRAWINGS

In order to better understand the present application, embodiments of the present application will be described in conjunction with the accompanying drawings. In the accompanying drawings:

FIG. 1 is a plan view of an embodiment of a compression dressing according to the present application;

FIG. 2 is a side view of the compression dressing of FIG. 1;

FIG. 3A is a plan view of a dressing according to some embodiments;

FIG. 3B is a side view of the dressing of FIG. 3A;

FIG. 4 is a plan view of a dressing according to some embodiments;

FIG. 5 is a plan view of a dressing according to some embodiments;

FIG. 6 is a plan view of a dressing according to some embodiments;

FIG. 7 is a plan view of a dressing according to some embodiments;

FIG. 8 is a plan view of a dressing according to some embodiments;

FIG. 9 is a plan view of a dressing according to some embodiments;

FIG. 10 is a side view of a dressing according to some embodiments;

FIG. 11 is a side view of a dressing according to some embodiments;

FIG. 12 is a side view of a dressing according to some embodiments;

FIG. 13 is a side view of a dressing according to some embodiments;

FIG. 14 is a side view of a dressing according to some embodiments;

FIG. 15A is a top view of a blister pack according to some embodiments;

FIG. 15B is a cross-section (A-A) of the blister pack of FIG. 15A;

FIG. 15C is a bottom view of the blister pack of FIG. 15A;

FIG. 16 is a schematic diagram of a colour coded kit of differently sized dressings according to some embodiments;

FIG. 17 is a cross-sectional view of a dressing dispenser according to some embodiments; and

FIG. 18 is a flow diagram showing a method of making, packaging and sterilising a bandage according to some embodiments.

### DESCRIPTION OF EMBODIMENTS

Embodiments described in this application are designed to be used for treatment of a wide variety of wounds and be substituted for dressings for simple traumatic wounds and for more complex and larger wounds. Embodiments can also be used as a compression bandage for application to provide first aid for envenomation or crush injury, as a dressing for chronic wounds and as a support bandage for injury. Dressings such as band aid type bandages, combinations of wound contact materials, absorbent materials, sticking or non-sticking binding materials and fastening materials and single piece, multiple component dressings can be replaced by one or more embodiments described herein.

Some embodiments relate to a wound dressing that is a multi-layer, self-adhering elastomeric bandage, which may be compressively wrapped around a wound or injury and has many benefits in wound treatment. Attached to the dressing bandage is a pad made from an absorbent material with a layer of non-stick material for placement over a wound. The bandage can be constructed in various sizes to cater for the size/type of wound and can be used for simple or complex wounds. The dressing can also be used to provide compressive force for first aid of envenomation or crush injury or as support for a musculoskeletal injury.

According to the present application, there is provided a compression dressing, which is referred to herein below as dressing.

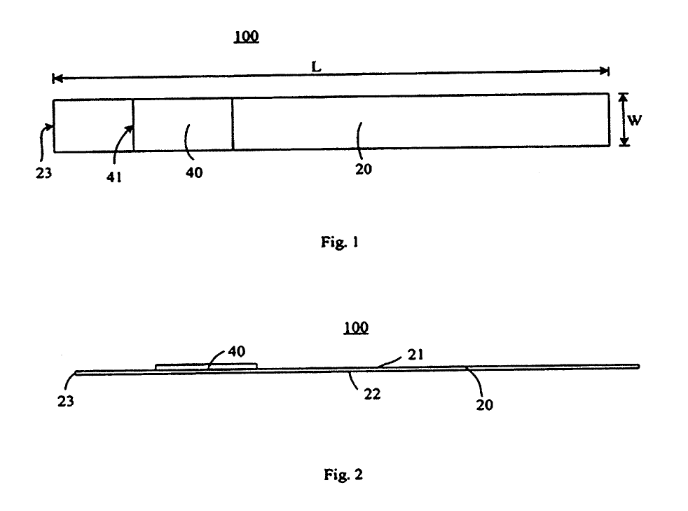

Referring to FIGS. 1 and 2, an embodiment of the dressing 100 comprises an elastic bandage strip 20 and an absorbent pad 40. Strip 20 is designed for exerting a compressive force when wrapped around a body part, sufficient to hold the compression dressing in place for a period of time to provide a therapeutic effect to a wound.

Pad 40 is affixed to an inner side 21 of a terminal end 23 of strip 20. The pad 40 may be affixed to the strip between 30 mm and 70 mm from the terminal end 23. A portion of strip 20 which is not covered by absorbent pad 40 is adapted to directly contact an outer side 22 of strip opposite of absorbent pad (i.e., a back side of strip) when dressing 100 is wrapped around the wounded body part.

The elastic bandage strip 20 can be self-adhering, so that this contact allows dressing 100 to self-adhere. i.e., to adhere to itself, and prevents sliding or shifting of the wrap after it is in place, without the use of a fastening mechanism. For most of the length of strip 20, absorbent pad 40 does not cover strip in order to provide a large, continuous contact area of the wrapped portion of strip.

Preferably, the adhesive side of the self-adhering elastic strip 20 is positioned on the outer surface 22 of the dressing 100, and a non-adhesive covering (not shown) can be provided for the exposed surface of the absorbent pad.

Preferably, the elastic bandage strip 20 according to the present application will provide a compressive force of between about 1 to 180 mm Hg in practice actual pressures averaging about 40 mm Hg are exerted when wrapped around a body part. This pressure is generally sufficient to provide a therapeutic benefit without necessarily restricting arterial and/or venous blood flow to a point distal to the bandaged body part such as a point of ischemia. Therapeutic benefits provided by the present application include control of bleeding, clot promotion, prevention of infection, therapeutic healing effects and approximation of wound surfaces.

Preferably, the self-adhering elastic bandage strip 20 is designed not to adhere to clothing, hair, skin or latex gloves.

Preferably, the self-adhering bandage strip 20 is constructed so as to have self-adhesive properties sufficient to cause two adjacent layers of the bandage to remain adhered to one another without the use of a fastening mechanism.

Of course, though the above self-adhering bandage strip 20 can be used without the use of a fastening mechanism, it is possible that the bandage further includes a fastening mechanism such as a clip or adhesive at the terminal end.

Materials suitable for use in elastic bandage strip 20 of the present application include materials which are elastic, conformable, provide adequate compression and which are self-adhering or which are made self-adhering by the addition of an adhesion surface thereon. Materials which may be sterilized, including ethylene oxide sterilized and radiation sterilized, are preferred.

In another preferred embodiment of the present preferably, the self-adhering bandage strip is constructed of woven elastomeric fibres. Alternatively, the self-adhering bandage strip is constructed of non-woven elastomeric fibres.

The two commonly used terms to describe these types of bandages are cohesive cotton bandage and non-woven cohesive bandage. Commercially available examples of elastic materials suitable for use in the present application include woven bandages such as CUTTER-WRAP self-adhesive bandage (Cutter Animal Health, Miles Laboratories, Inc., Shawnee, Kans.), MEDI-RIP self-adherent bandage (Conco Medical Company, Bridgeport, Conn.) or SELF-GRIP sports tape/bandage (LMA, Ltd., South Norwalk, Conn.) and nonwoven bandages such as ROFLEX cohesive flexible bandage (Smith and Nephew Rolyan Inc., Menomonee Falls, Wis.), VET-FLEX veterinary flexible bandage (The Butler Company, Columbus, Ohio), CO-FLEX cohesive flexible bandage (Andover Coated Products, Inc., Marblehead, Mass.), FLEXUS support wrap (Kimberly-Clark Corporation Animal Care Division, Roswell, Ga.), COBAN self-adherent wrap (3M, St. Paul, Minn.), MDS (Suzhou Medsport Products Co., Ltd) and other cohesive bandages.

These examples are intended to illustrate the function of the bandage strip of the application and to outline some of the differing brands available and varying techniques used in their manufacture. For the purpose of this application the type of cohesive bandage used is to be of either non-woven or cotton cohesive type with any or all brand or manufacturing techniques not exclusive to the above being used.

A material made of melt blown microfiber webs may also be used in the strip 20 of the present application. The melt blown microfiber webs may be composed of a variety of well-known thermoplastic elastomers including polyurethane, polystyrene-isoprene block copolymer, styrene-butadiene block copolymer, (KRATON polymer, Shell Oil Company, Belpre, Ohio) and blends of these elastomers with polyolefins such as polypropylene and polyethylene. In addition, the melt blown microfiber webs may include, but are not limited to, staple fibres, such as rayon, polyester, nylon, cotton, LANSEAL fibre, cellulose, or polypropylene fibres, to provide a blend of elastomeric and staple fibres.

The absorbent pad 40 is attached to the self-adhering elastic bandage strip 20 by an attaching method such as, but not limited to, stitching, welding, needle tacking or bonding.

Suitable adhesives which may be used to bind an absorbent pad 40 to THE elastic bandage strip 20 of the present application include pressure sensitive adhesives such as polyacrylates, polyvinyl ethers, and poly alpha-olefins, as well as polymers which may be formulated with appropriate tackifiers such as natural rubber, stylene-isoprene block copolymer, silicone rubber, cis-polyisoprene, styrene butadiene, and cis-polybutadiene, hot-melt adhesives such as low-density polyethylene, ethylene-vinyl acetate copolymers, ethylene-ethyl acrylate copolymers, paraffin waxes, polyesters, polyamides, polypropylene, styrene-butadiene block copolymers and polyurethanes, and curable adhesives such as silicones and urethanes. In addition, repositionable adhesives such as microsphere pressure sensitive adhesives may be used to bond the absorbent pad to the elastic bandage strip. Highly preferred adhesives would be those which are biocompatible with skin and which generally do not cause irritation or undesirable sensitivity reactions when in contact with skin for extended periods of time under normal conditions such as natural rubber and acrylate based adhesives.

Preferably, the absorbent pad 40 is constructed of woven or non-woven material of natural or synthetic fibres, such as, but not limited to, rayon, polyester, polyurethane, polyolefin, cellulose, cellulose derivatives, cotton, orlon or hydrogel polymeric materials. Preferably, the absorbent material both absorbs wound exudate and protects the wound by absorbing shocks. Other types of materials having similar absorbent properties and characteristics would also be suitable for use in this application. Examples of absorbent materials are dacron-polyester cast padding, (3M, St. Paul, Minn.), DELTA-ROL acrylic cast padding 6884, (Johnson & Johnson, New Brunswick, N.J.), SOF-ROL 100% needle-tacked rayon cast padding HRI 8137-009034, (Johnson & Johnson, New Brunswick, N.J.), SPECIALIST cotton cast padding HRI 8137-009044 (Johnson & Johnson, New Brunswick, N.J.), WEBRIL cotton undercast padding 3175 (The Kendall Company, Boston, Mass.), WEBRIL II cotton undercast padding 4221 (The Kendall Company, Boston, Mass.), nonwoven cotton web 142-451 and nonwoven rayon/polyester web 140-037 (VeraTec Company, Wapole, Mass.), and an absorbent resilient open-cell foam such as polyurethane, polyester, polyether, polyolefin foams.

The surface of the absorbent material which contacts the wound may additionally be treated or modified so that it will not adhere to the wound. For example, the absorbent material may be covered with a variety of commercially available wound contact materials such as TEGAPORE woven nylon web, TEGADERM polyurethane film or TEGASORB hydrocolloid (all available from 3M, St. Paul, Minn.) as well as other well-known related materials.

Preferably, the compression dressing 100 is rolled into a roll and packaged in a sterile packaging. The dressing 100 may be sterilized by, for example, exposure to heat, radiation or gas.

Preferably, the absorbent pad 40 has a width equal to or smaller than that of the elastic bandage strip 20.

Preferably, a distance between a terminal end 41 of the absorbent pad 40 and the terminal end 23 (e.g., the left side) of the elastic bandage strip 20 is from 10 mm to 100 mm. More preferably, the distance between the absorbent pad and the terminal end of the elastic bandage strip is from 50 mm to 70 mm. For example, the length (L) and width (W) of the elastic bandage strip 20 can be 240-260 mm and 25 mm, the length and width of the absorbent pad 40 can be 25 mm and 25 mm, and the distance between the terminal end 41 of the absorbent pad 40 and the terminal end 23 of the elastic bandage strip 20 can be 25 mm. The length (L) and width (W) of the elastic bandage strip 20 can be 450-500 mm and 25 mm, the length and width of the absorbent pad 40 can be 50 mm and 25 mm, and the distance between the terminal end 41 of the absorbent pad 40 and the terminal end 23 of the elastic bandage strip 20 can be 50 mm. The length (L) and width (W) of the elastic bandage strip 20 can be 1000-1200 mm and 50 mm, the length and width of the absorbent pad 40 can be 100 mm and 50 mm, and the distance between the terminal end 41 of the absorbent pad 40 and the terminal end 23 of the elastic bandage strip 20 can be 70 mm. The length (L) and width (W) of the elastic bandage strip 20 can be 1000-1200 mm and 75 mm, the length and width of the absorbent pad 40 can be 150 mm and 75 mm, and the distance between the terminal end 41 of the absorbent pad 40 and the terminal end 23 of the elastic bandage strip 20 can be 70 mm. The length (L) and width (W) of the elastic bandage strip 20 can be 1000-1200 mm and 100 mm, the length and width of the absorbent pad 40 can be 200 mm and 100 mm, and the distance between the terminal end 41 of the absorbent pad 40 and the terminal end 23 of the elastic bandage strip 20 can be 70 mm. The length (L) and width (W) of the elastic bandage strip 20 can be 2000-2200 mm and 150 mm, the length and width of the absorbent pad 40 can be 200 mm and 150 mm, and the distance between the terminal end 41 of the absorbent pad 40 and the terminal end 23 of the elastic bandage strip 20 can be 70 mm. It should be understood that, the present application is not limited to the size specifications above, and any other size specifications dependant on the specific usage of the bandage will be also appropriate.

According to yet another aspect of the present application there is provided a method of preparation and packaging of the compression dressing to enable easier application. This method is effected by rolling and packaging the complete dressing loosely to prevent self-adherence of the cohesive bandage after manufacture and whilst in the packaging.

When prepared and packaged in this manner, the compression dressing 100 may be applied around a wounded body part without difficulty caused by excessive adhesion of the cohesive bandage, affording more rapid and effective treatment of a wound.

Some potential uses for the dressing include:
For wounds both acute and chronic; For open and closed wound's;
For Lacerations, abrasions, penetration wounds, gunshot wounds, surgical wounds, thermal wounds, chemical wounds, electrical wounds, avulsions;
For arresting of uncontrolled haemorrhage;
For dressing of wounds, both acute and chronic, to promote healing;
For to promote healing in long term wounds with regular changing (i.e. wounds on the legs caused by body fluid excess; commonly called venous ulcers);
For small wounds to fingers or toes;
As a dressing for bites, snake bites or stings where compression of the affected limb is required to minimize the effects of envenomation due to poison entering the systemic circulation;
As a dressing for wounds through which irrigation and cooling can be performed such as burns; and
As a dressing for the use of compressive force in the instance of prolonged compression of a limb causing tissue damage (commonly known as “crush injury”) to be applied before release of the damaging compressive force.

A wide variety of materials have been used to treat by dressing different types of wounds. Typical materials that are used in the various dressings include individual wound contact materials, absorbent materials, adhesive binding and non-adhesive binding materials and fastening materials. Dressings are also available that integrate these components and are manufactured as a single item. These range from small dressings such as adhesive band-aid types and similarly constructed larger adhesive dressings (Henceforth these are referred to as “adhesive bandages”). These dressings can provide beneficial conditions to promote wound healing or create therapeutic effect by compression, however the standard materials used in them have some limitations and deficiencies in achieving their desired effect.

The use of integrated pieces of wound pad attached to a glue-covered strip for wounds (adhesive bandages) has the limitation that the adhesion, and thus longevity, of the bandage can be poor. Wound exudate or moisture/perspiration can weaken the adhesion of the glue in this type of dressing leading to failure of the dressing to remain secure for the intended length of time. Similarly, dressings constructed of cotton or linen binding materials are only fastened at their terminal end and wound exudate or moisture/perspiration can weaken the adhesion of the fastening device (most commonly sticking tape) which secures them.

Adhesive bandages are often constructed of plastic or woven fabric covered in glue adhesive. Construction with this material poses a technical problem as it often fails to conform to the anatomy during movement, causing disruption of glue adhesion and failure of the dressing. Larger self-adhesive dressings have a larger fastening surface area but the limitations in flexibility or conformity during movement and in the presence of moisture remain. The relatively rigid construction of larger self-adherent bandages also causes the technical problem of a lack of comfort to the wearer. The binding material is rigidly fastened to itself and the wearer's skin/hair along its length and does not conform elastically to movement. This causes difficulty/pain in movement. With natural movement of self-adhesive bandages glue integrity is easily disturbed and the dressing fails because of this.

The fastening qualities of self-adherent bandages (such as Band-Aid type bandages and larger types) also have the undesirable limitation of leaving glue residue on the users skin, which is unsightly and can contaminate a wound (with glue residue or particles attached to them) with detrimental healing effects. In addition, self-adherent dressings stick to skin, hair and wounds and are painful to remove.

It may be possible to address adhesion failure due to movement, moisture or rigidity of the dressing by creating a dressing, which has strong adhesion, is resistant to release in the presence of moisture and can flexibly conform to the wearer.

It may be possible to avoid the dressing sticking to skin, hair and wounds causing pain, by creating a dressing that does not adhere to skin, hair or wounds but will still adequately fasten to itself.

The problem of glue residue left on skin and the associated contamination of wounds may be addressed by creating a dressing which does not adhere using glue.

The cohesive bandage used in the described embodiments can adhere to itself along its entire length without sticky/tacky glue and it maintains this adhesion in the presence of wound fluid/exudate and in moist environmental conditions. The cohesive bandage used in the dressing of the described embodiments may be constructed with a natural or synthetic strip material coated with coherent binding agents, for instance, hypoallergenic natural rubber or synthetic polymers. This construction gives the binding material the property of adherence to itself when the coherent binding agents on the inner layer of bandage contact the binding material of the outer layer. The cohesive bandage used in the described embodiments may also be more flexible and elastic materiel in comparison to adhesive bandages. The cohesive bandage may be able to flex from the applied shape and remain adhered. For example, when the dressing is applied circumferentially over a joint such as a knuckle or elbow, this joint can be flexed/extended and the dressing may be able to conform without losing adhesion. The qualities of flexibility and adhesion that cohesive bandage has allow adhesion to persist in moist conditions more effectively than self-adherent bandages which may lose glue integrity due to flexing.

The flexibility and adhesion qualities of the cohesive bandage used in the dressing of the present application may also solve the technical problem of adhesion failure due to movement of the wearer. The available surface area for adhesion in the dressing may also be much larger compared to conventional cotton rolling bandages which are only fastened at their terminal end and do not adhere along their length. The properties of adhesion in moist environments, flexibility of the binding material, large fastening surface area and comfort for the wearer combine to offer significant technical advantages over known self-adherent and non-adherent binding materials.

The cohesive bandage used in the dressing of the present application may not contain any sticky/tacky glue. It may not adhere to the wearer at all. This may be beneficial in comparison to adhesive wound bindings, as skin, hair or wounds are unlikely to be contaminated with glue residue or particulate matter stuck to this glue. The adhesion properties of the cohesive bandage also prevent the adhesion of the dressing to the person applying it, which may increase ease of use and decrease the time needed to apply a wound dressing. Similarly, because the invention does not adhere to skin, hair or wounds, removal of the dressing may be easier and involve less pain for the patient.

Adequate protection of a wound in padding combined with keeping a wound dressing of an appropriate size is a technical problem for adhesive bandages. Generally there is very little padding in the construction of the binding material of adhesive bandages due to their construction with plastic or thin woven materials. When using adhesive bandages as constructed in the above manner, the wearer can experience pain and trauma to a wound if accidently knocked.

Similarly, cotton or linen bandages have limited protective properties from accidental knocks or bumps. While they offer more protection than adhesive bandages they are generally of a thin woven cotton or linen construction. A layer of padding is often added to the component parts of a wound dressing achieve adequate padding characteristics in the dressing.

Poor padding characteristics in self-adherent bandages and multi-component bandages may be addressed by creating a dressing which offers more padding while maintaining minimal dressing bulk.

The dressing of the present application may provide padding/protection for the wearer in the construction of the absorption/wound contact pad. In addition, the bandage itself offers additional padding due to its construction. The cohesive bandage used in the construction of the dressing of the present application is a thin construction of either meltblown synthetic or woven fibers which create an undulating surface. The constructed fibers and adhesive also have the property of elasticity. These two factors of construction combine to create a binding material which is soft and can also absorb more energy from accidental knocks than woven cotton or linen bandages. In addition, when the bandage is not stretched to the full extent, it may provide further padding.

Timely and physically easy application of a dressing is advantageous in achieving optimal therapeutic effect. The use of multiple individual components in dressings (non-stick wound contact materials absorbent pads and/or padding materials, bandaging/binding and fastening materials) creates a technically/physically difficult and time-consuming method of dressing application.

The technical/physical steps required to apply a dressing of this type or similar types of dressing are as follows:
Individual packages are opened (≧3);
Individual components are laid out on an available surface to be individually picked up and applied;
The wound pad is placed over the wound and held in place with one hand;
The other hand picks up and partially unfurls the rolled binding material;
While securing the pad from movement from desired placement the same hand simultaneously grasps the end of the binding material to apply over the pad and begin unfurling it in a circumferential manner;
Circumferential wrapping continues and desired tension to the binding material must be maintained to prevent slipping/loosening;
To complete the dressing the user secures the binding at its terminal end with adequate pressure to maintain compression along the entire bandage with one hand;
the user then picks up and applies a fastening device (such as sticking tape or metal hooks) using the other hand.

The complexity of the task (in motor skill and time required) is relatively high and various deleterious effects to the application of an optimal wound dressing can occur as a result of this complexity.

Due to the difficulty of holding the pad in place and grasping the terminal end of the binding material with the same hand, the absorbent pad and wound contact materials can shift off the wound as circumferential binding begins. This misplacement can prevent the therapeutic efficacy of wound contact materials to absorb blood/fluid and promote healing. Misplacement can also cause adhesion of a wound to binding materials and pain when the dressing is removed.

Slipping/loosening of a compression bandage can reduce the therapeutic effect. For example in the instance of envenomation, loss of tension can cause life-threatening migration of toxin to the systemic circulation and/or through the lymphatic system. Blood loss due to ineffective compression can also occur. Tension loss in applying a multicomponent dressing can easily occur due to the physical difficulty of the task and lack of adhesion of binding materials. The application of a multi-component dressing in less than ideal conditions, by an inexperienced user or a user who has poor dexterity exacerbates the technical difficulties inherent in applying an effective multicomponent dressing.

It may be possible to reduce the high time requirements and physical complexity of applying a multicomponent dressing by eliminating the requirement to open multiple items from packaging to create a complete dressing, to reduce the individual steps to apply the dressing, and to reduce the dexterity required to apply the dressing.

Wound contact pad movement and undesired tension loss when applying a dressing may be addressed by creating a dressing that allows ease of physical control in applying the circumferential layers of binding and creating a dressing that maintains desired tension on each layer of circumferential binding and thus cannot move or slide if the user loses control of the binding.

The dressing of the present application may replace a non-stick wound contact material, an absorbent pad, bandaging/binding material and a fastening material with one single item, so that only one package needs to be opened to apply a complete dressing. The described dressing is far easier to apply than a multi-component dressing.

Example technical/physical steps required to apply the dressing of the present application are as follows:
Individual package is opened and the dressing remains in the user's hand;
The wound pad and the end of the binding material is placed over the wound held by one hand;
The other hand holds the rolled binding material;
Circumferential application begins;
Pad movement is minimised as the bandage adheres to itself without movement during circumferential application and will not slip/slide/lose desired tension;
Upon reaching the desired binding length the dressing remains fastened to itself without picking up and applying adjunct fastening devices, and if required the dressing may be torn vertically by hand when the desired length is less than the dressing length.

The component parts of existing wound dressings are often contained in multiple individual packages. All packaging must be opened before use. This results in a large amount of refuse for disposal.

The problem of excess refuse of dressing packaging may be addressed by creating a dressing which requires less packaging.

The dressing of the present application may be enclosed in a single sterile package, since all component parts of the dressing are integrated. This means that packaging waste can be reduced in comparison to multi-component dressings. The described dressing may also application time to be reduced for the user as only one package needs to be opened.

There may also be a cost saving in packaging a single dressing including all of the necessary components in comparison to the expense of using individually wrapped wound contact material, absorbent material, padding material, binding material and fastening material.

The sterility of the component parts of a dressing is important as introduction of contaminants to a wound can cause infection or delayed healing. It is technically difficult to maintain sterility of separate dressing components before application without ideal conditions such as a space prepared as a sterile area. Individual dressing components need to be set down on an available space before sequential application and can become contaminated due to this. It is technically difficult to maintain sterile conditions in applying a dressing comprised of multiple components without aids and conditions commonly found only in clinical environments.

The problem of potential contamination of individual dressing components before application may be addressed by creating a dressing that does not need a sterile area to set up and does not need its components to be set down before use.

The dressing of the present application can be removed from its packaging and directly applied to the patient. No component parts need to be set down to apply it and it may be easier to maintain its sterility is maintained before application to the wearer. This may be particularly beneficial when a dressing needs to be applied in less than ideal conditions where sterile fields for setup are not present. For example in pre-hospital environments including outdoors, in private residences or business, sports events etc.

The accurate measurement of fluid loss from a wound can be beneficial for health care workers and patients. It is clinically useful to have an accurate measurement of blood/fluid loss to calculate fluid resuscitation requirements, to monitor the amount of wound exudate and wound healing progress or to record body fluid input vs. output. It is technically difficult to calculate the amount of blood/fluid loss from a wound when dressed with multicomponent dressings as the number of components and their individual absorbency characteristics vary.

The problem of incalculable fluid loss from wounds due to varying wound dressing components may be addressed by creating a dressing which can contain a known quantity of fluid.

The dressing of the present application may come in standard sizes with each wound pad holding a known quantity of fluid for each size. In addition the bandage used in the dressing may be non-porous in comparison to cotton rolling bandage and the fluid lost from a wound may largely be contained within the wound pad itself. Due to this construction the dressing may provide a technical advantage over multi-component wound dressings as fluid loss can accurately be calculated and this measurement is transferrable on subsequent applications of the same size dressing. This may allow more accurate estimation of blood loss and the volume of wound exudate. This aspect of the dressing of the present application may aid clinicians in calculating volume loss and/or resuscitation requirements and healing progress as exudate decreases.

The use of highly porous binding components for the outer layer of dressings poses a technical problem in containment of bio hazardous waste and infection control. Commonly, outer binding dressing material is made from highly absorbent materials such as cotton bandage. With use of these, blood or fluid can migrate from the wound to the outer layer of the dressing and come into contact with health care workers or the surrounding environment easily.

The problem of containment of bio-hazardous waste from dressing binding material may be addressed by using a non-porous binding material which may not soak up much (if any) wound fluid/exudate.

The dressing of the present application uses cohesive bandage which is far less porous in comparison to cotton/linen bandage. Wound fluid/exudate may be less likely to migrate into it as the cohesive bandage of the described dressing has a lesser absorption potential than cotton or linen bandages. Further, the wound pad of the dressing may have a large size and absorb a known quantity of fluid/exudate. This may allow the expected exudate from a wound to be predicted and the dressing can be changed before the wound binding materials become soaked thereby lessening the potential of bio-hazardous waste migrating from a wound to the surrounding environment.

Commonly used non-adherent binding materials in dressings have a tendency to move/slide or loosen after application. The technical problem is that non-adherent or elastic-binding materials can change their placement and that targeted compression pressures or coverage of the dressing over the affected area can fail.

Non-adherent binding materials can slide/loosen/move due to a loss of elasticity of the binding material, due to movement by the wearer or from loss of grip forces in the stretched binding material (contact dynamics/mechanics). The amount that binding materials can slide/loosen/move can also be affected by moisture permeating the binding. This can cause deleterious effects. For example a compression dressing for snake envenomation can lose grip over time causing uptake of venom to the systemic circulation and/or lymphatic system. Or the dressing for a crush type injury can loosen releasing toxic substances (potassium, myoglobin, phosphorous etc.) causing renal failure or cardiac arrest.

Adhesive bandage bindings also have a tendency to move/slide or loosen in the presence of moisture from the wound or environment with the same loss of compression and coverage as outlined above. Further adhesive fastenings such as glue or sticking tape are far less effective in wet conditions that can be found in the pre-hospital environment.

Multicomponent wound dressings can be secured using metal hooks each end of an elastomeric strip to achieve desired time in situ however loss of desired compression and coverage characteristics can still occur. Securing a dressing with metal hooks, sticking tape or other fasteners at its terminal end does not prevent the bandage from slipping/sliding and changing the desired uniform compression characteristics of a dressing.

The problem of the tendency of dressings to move/slide or loosen after application may be addressed by creating a dressing which adheres along its entire length without the negative qualities of glue adhesive.

The problem of the tendency of dressings to move/slide or loosen after application due to moisture may be addressed by creating a dressing which self-adheres despite the presence of moisture.

The problem of the tendency of dressings to move/slide or loosen after application due to fastening only at the terminal end may be addressed by creating a dressing which adheres along its entire length which may allow targeted compression and coverage to be maintained.

The property of self-adhesion of each circumferential layer of cohesive bandage used in the dressing reduces or eliminates movement/sliding after application. In application of the dressing, uniform or targeted compression can be achieved as each layer adheres to the one below it and thus compression and placement is maintained. The dressing may achieve this without the use of glue as a fastening agent.

The cohesive bandage of the dressing of the present application adheres along its entire length and may not slide or loosen as a result of inadequate fastening and redistribution of compressive forces. This may provide a benefit in comparison to non-adherent bandages of cotton or linen construction which adhere only at the terminal end and will move over time.

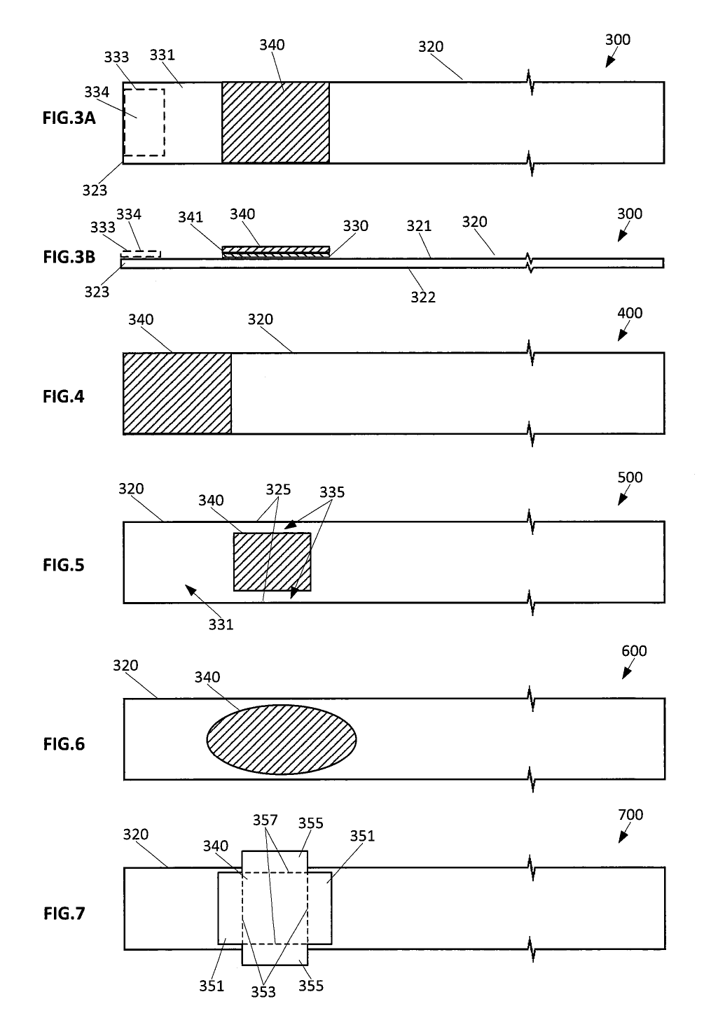

Referring to FIGS. 3A and 3B, a wound dressing 300 is shown according to some embodiments. The dressing 300 comprises a cohesive or self-adhering bandage 320 and an absorbent pad 340.

The bandage 320 has a length, width and thickness, and may define a cohesive bandage strip. The cohesive bandage 320 may be formed of a material which can adhere or cohere to itself, without strongly adhering to other surfaces such as a patient's wound, skin, hair, or other tissue, for example. The bandage 320 may be elastic along its length and cohere to itself with sufficient strength that, when wrapped around a patient's appendage, limb or torso, it can provide a compressive force through circumferential tension which is sufficient to provide a therapeutic benefit. The cohesive bandage 320 can adhere to itself, as it is wrapped around a body part and back onto itself, such that the bandage 320 can hold the dressing in place without the use of any additional fasteners. Various cohesive bandages are commercially available such as described above.

The pad 340 has a length, width and thickness. The pad 340 may also have a known absorptive capacity to assist in calculating fluid loss from a wound. The pad 340 may be formed of any suitable absorbent material such as non-woven cotton fibres or other natural or synthetic fibres, for example. In some embodiments, the absorbent material may be encased or sandwiched between two layers of film or porous material.

In some embodiments, the pad 340 may be treated, doped or impregnated with various additives to provide further beneficial therapeutic effects. Additives may include any one or more of: antimicrobials, antibiotics, antibacterial substances, antiseptics, haemostatic substances, coagulants, clotting agents, gels, hydrogels, analgesics, anaesthetics, anti-inflammatories, antihistamines, drugs, ointments, creams and medication. Haemostatic agents which may be used include: plant (cellulose)-based, gelatin-based, platelet rich plasma, collagen-based, fibrin-based, thrombin-based, chitin/chitosan-based and mineral-based agents. Some possible additives are silver derivatives, iodine derivatives, petroleum derivatives, saline, chitosan, hydrofibres, hydrogels, alginate, Melaleuca oil, sphagnum moss or substances derived therefrom, antimicrobial oils and honey. Silver, iodine and sphagnum moss derivatives can provide an antimicrobial effect. Silver derivatives in particular may enhance chronic wound care, especially where the microbial burden appears to be delaying the healing process. Petroleum derivatives can provide an analgesic effect. Saline can help maintain moisture and provide a cooling effect. Chitosan is a shellfish derived substance which, when it comes in contact with blood, swells, gels, and sticks together to make a gel like clot. Hydrofiber/gel dressings gel upon contact with moisture, locking in fluid that is absorbed by the dressing. In this manner, they help to maintain a moisture balance in the wound bed that is not too wet or too dry, protecting the edges of wounds from becoming macerated. Hydrogels can be effective in treating burns such as superficial or partial thickness burns. A suitable hydrogel may comprise Melaleuca oil, water and a gelling agent. Melaleuca oil provides analgesia, cooling and antimicrobial properties when applied to wounds; it is particularly useful in treating burn wounds. The high osmolarity of certain honeys can restrict the growth of bacteria and encourage healing. Honey may also provide some antimicrobial effect.

In some embodiments, the dressing 300 may comprise a cold pack or ice pack. In some embodiments, the cold pack may be disposed between the bandage 320 and the pad 340, and may be affixed to the dressing 300. In some embodiments, the dressing 300 may comprise a sleeve, slot, pouch or pocket sized to receive the cold pack, and the cold pack may be removably placed in the pouch or may be sewn into the pouch.

Dressings 300 including a cold pack may be stored below ambient temperature in order to provide a cooling effect on application, or alternatively, the cold pack may be an “instant” type cold pack that makes use of an endothermic chemical reaction to provide the cooling effect. One example of such an “instant” type cold pack comprises two bags; a first outer bag containing ammonium nitrate, calcium ammonium nitrate or urea, and a second inner bag inside the first, the inner bag containing water. When the inner bag of water is broken by squeezing the pack, it is allowed to dissolve the solid in an endothermic reaction. This reaction absorbs heat from the surroundings, quickly lowering the temperature of the pack. Such a cooling effect reduces the immediate area of surface temperature which may enhance vascular constriction, limit blood flow, reduce inflammation and swelling, and provide some pain relief.

The pad 340 is affixed to the bandage 320 which may be more convenient for users when applying the dressing to a patient. The pad 340 may be affixed to the bandage 320 at or near a terminal end 323 of the bandage 320. In some embodiments, a terminal end 341 of the pad 340 may be located at the terminal end 323 of the bandage 320, as shown in the dressing 400 of FIG. 4. In other embodiments, there may be an end margin or tail 331 between the terminal end 323 of the bandage 320 and the terminal end 341 of the pad 34, as shown in dressing 300 of FIG. 3A and in the dressings 500, 600, 700, 800, and 900 of FIGS. 5 to 9, respectively. In some embodiments, sides 345 of the pad 340 may be located at sides 325 of the bandage 320, as shown in dressings 300 and 400 of FIGS. 3A and 4. In other embodiments, there may be side margins 335 between the pad sides 345 and the bandage sides 325, as shown in dressing 500 and 600 FIGS. 5 and 6. Similar side margins 335 may be included in dressings 300 and 400.

The pad 340 may be affixed to an inner surface 321 of the bandage 320. The inner surface 321 may be tackier or stickier than an outer surface 322 of the bandage 320, which may be rougher. The pad 340 may be affixed to the bandage 320 by way of bonding, heat bonding, welding, stitching, tacking or with adhesive 330, as shown in FIG. 3B. The adhesive 330 may be flexible, waterproof and hypoallergenic.

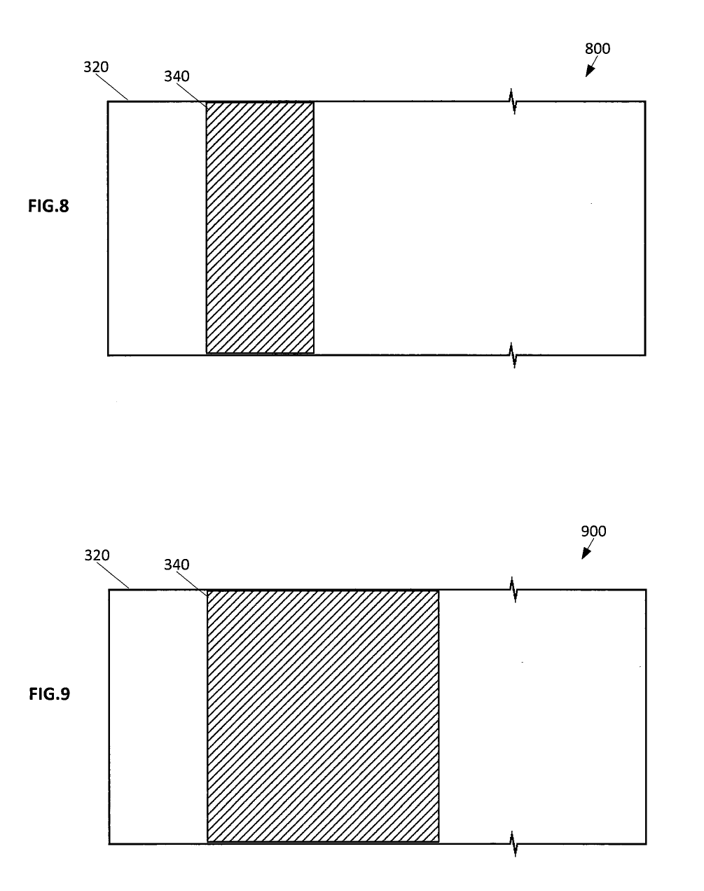

The pad 340 may define any suitable shape. For example, in some embodiments, the pad 340 may be round or have rounded corners, as shown in dressing 600 of FIG. 6. The dressings 300, 400 and 500 may also include a pad 340 with rounded corners, in some embodiments. In other embodiments, the pad 340 may comprise flaps or wings to fold out and increase the surface area presented to the wound, as shown in dressing 700 of FIG. 7. The pad 340 may comprise end wings 351 to unfold longitudinally about fold lines 353 along the bandage 320. Alternatively, or in addition, the pad 340 may comprise side wings 355 to unfold laterally about fold lines 357 over the bandage sides 325, as shown in dressing 700 of FIG. 7. In some embodiments, the pads 340 of dressings 300, 400, 500 and 600 may include end wings 351 and/or side wings 355.

The absorptive capacity or absorbency of the pad 340 may be between about 20% and 200% of the dry volume of the pad 340, optionally about 50-150%, optionally about 60-100%, optionally about 70-90%, optionally about 80%. The absorptive capacity of the pad 340 may be between about 0.1 mL/cc and 1.5 mL/cc of liquid such as blood or exudate, optionally about 0.5-1.2 mL/cc, optionally about 0.8-1 mL/cc, optionally about 1 mL per cubic centimetre. The total absorptive capacity of the pad may be between about 1 mL and 200 mL, optionally about 2-5 mL, optionally about 2.5-3.2 mL, optionally about 3.1 mL, optionally about 5 mL, optionally about 15-100 mL, optionally about 8-10 mL, optionally about 10 mL, optionally about 22-29 mL, optionally about 28.1 mL, optionally about 48-60 mL, optionally about 60 mL, optionally about 75 mL, optionally about 96-120 mL, optionally about 120 mL, optionally about 150-300 mL, optionally about 300 mL, optionally about 300-450 mL, optionally about 400 mL, optionally about 500-700 mL.

The dressing 300 may be sized to suit different applications, and may have any suitable dimensions. The shape and dimensions of the bandage 320, pad 340, margins 331, 335, and wings 351, 355 may be adjusted to any suitable shape, size and proportions for different applications. Some possible examples are shown in FIGS. 3 to 9.

The tail 331 may be long enough to allow a user to position and hold the pad 340 in place on a body part when beginning to apply the dressing 300 without needing to touch or disturb the wound, which may be desirable in some applications. The tail 331 may be short enough that it does not become a hindrance to the user by bunching up, being difficult to manage, or wrapping around the body part and onto the wound. Different tail lengths may be more appropriate for different sized dressings and/or in different applications.

In some embodiments, the dressing 300 may further comprise a tag or tab 333 disposed at the terminal end 323, as shown in FIG. 3A. The tab 333 may assist a user in finding the end 323 of the dressing 300 when it is in a rolled or folded configuration. The tab 333 may assist a user in peeling the tail 331 of the dressing 300 up from the rest of the rolled or folded dressing 300 making it easier to unroll or unfold the end 323 from the rest of the dressing 300. The tab 333 may comprise a non-adhesive, non-cohesive material or any material with an outer surface 334 that will not self-adhere to the outer surface 322 of the bandage 320. Some possible materials for the tab 333 include synthetic fibres, natural fibres, woven fibres, non-woven fibres, cotton and biodegradable material, for example. The tab 333 may be affixed to the bandage 320 by way of bonding, heat bonding, welding, stitching, tacking or adhesive bonding with a flexible, waterproof and hypoallergenic adhesive. The tab 333 may be affixed to the terminal end 323 so as to extend from the terminal end 323 away from the bandage 320. Alternatively or additionally, the tab 333 may be disposed to cover an area of the bandage 320. The tab 333 may extend across a partial width of the bandage 320, or in some embodiments, may extend across the entire width of the bandage 320. In some embodiments, the dressings 400, 500, 600, 700, 800 and 900 may comprise a similar tab 333.

Differently sized dressings 300 may comprise various combinations of differently sized pads 340 and differently sized bandages 320. The dressing 300 may be sized to wrap various body parts of an adult human patient, a child or a different animal. For example, the dressing 300 may be sized to wrap around a digit, finger, thumb, toe, limb, arm, leg, head, neck or torso. Differently sized pads 340 may also be suited to treating different wounds and for different therapeutic applications.

The length of the bandage 320 is substantially greater than the width of the bandage. The stretched length of the bandage 320 may be substantially greater than the relaxed length. The stretched length of the bandage 320 may be between about 110% to 400% of the relaxed length, optionally about 150-250%, optionally about 200%. The relaxed length of the bandage 320 may be between about 100 mm and 3000 mm, optionally about 200-300 mm, optionally about 240-260 mm, optionally about 250 mm, optionally about 400-550 mm, optionally about 450-500 mm, optionally about 500 mm, optionally about 700-800 mm, optionally about 750 mm, optionally about 800-1400 mm, optionally about 1000-1200 mm, optionally about 1000 mm, optionally about 1500-2500 mm, optionally about 2000-2200 mm, optionally about 2000 mm. The width of the bandage 320 may be between about 10 mm and 300 mm, optionally about 10-30 mm, optionally about 20-25 mm, optionally about 25 mm, optionally about 40-60 mm, optionally about 50 mm, optionally about 65-85 mm, optionally about 75 mm, optionally about 90-110 mm, optionally about 100 mm, optionally about 100-200 mm, optionally about 140-160 mm, optionally about 150 mm. The width of the bandage 320 may be between about 1% and 20% of the length of the bandage 320, optionally about 5-15%, optionally about 5-10%, optionally about 10%. The bandage 320, may have a relaxed or bunched thickness which is substantially greater than its stretched thickness. The bunched thickness may be between about 0.2 mm and 2 mm, optionally about 0.5 mm and 1.5 mm, optionally about 1 mm. The stretched thickness may be between about 0.01 mm and 0.1 mm, optionally about 0.03-0.08 mm, optionally about 0.05 mm. The bunched thickness may be between about 2 times and 100 times thicker than the stretched thickness, optionally about 5-50 times thicker, optionally about 10-30 times thicker, optionally about 20 times thicker.

The length of the pad 340 along the length of the bandage 320 may be between about 10 mm and 300 mm, optionally about 10-30 mm, optionally about 20-25 mm, optionally about 25 mm, optionally about 40-60 mm, optionally about 50 mm, optionally about 65-85 mm, optionally about 75 mm, optionally about 90-110 mm, optionally about 100 mm, optionally about 100-200 mm, optionally about 140-160 mm, optionally about 150 mm, optionally about 150-250 mm, optionally about 180-220 mm, optionally about 200 mm. The width of the pad 340 across the width of the bandage 320 may be between about 10 mm and 300 mm, optionally about 10-30 mm, optionally about 20-25 mm, optionally about 25 mm, optionally about 40-60 mm, optionally about 50 mm, optionally about 65-85 mm, optionally about 75 mm, optionally about 90-110 mm, optionally about 100 mm, optionally about 100-200 mm, optionally about 140-160 mm, optionally about 150 mm. The width of the pad 340 may be between about 25% and 400% of the length of the pad 340, optionally about 50-300%, optionally about 60-200%, optionally about 80-120%, optionally about 100%, optionally about 40-100%, optionally about 50-60%. The width of the pad 340 may be between about 50% and 200% of the width of the bandage 320, optionally about 75-120%, optionally about 100%. The thickness of the pad 340 may be between about 0.5 mm and 7 mm, optionally about 2-4 mm, optionally about 2 mm, optionally about 2.5 mm, optionally about 3 mm, optionally about 4 mm.

The length of the tail 331 may be between about 10 mm and 200 mm, optionally about 80-120 mm, optionally about 90-110 mm, optionally about 95-105 mm, optionally about 100 mm, optionally about 50-100 mm, optionally about 60-80 mm, optionally about 65-75 mm, optionally about 70 mm, optionally about 50 mm, optionally about 20-40 mm, optionally about 25-35 mm, optionally about 30 mm. The length of the tail 331 may be between about 1% and 10% of the length of the bandage 320, optionally about 5-10%, optionally about 6-8%, optionally about 7%.

The length of the tab 333 may be between about 2 mm and 200 mm, optionally about 50-100 mm, optionally about 60-80 mm, optionally about 65-75 mm, optionally about 70 mm, optionally about 50 mm, optionally about 20-40 mm, optionally about 25-35 mm, optionally about 30 mm, optionally about 2-20 mm, optionally about 5-15 mm, optionally about 10 mm. The length of the tab 333 may be between about 0.1% and 100% of the length of the tail 331, optionally about 1-20%, optionally about 2-10%, optionally about 4-8%, optionally about 7%, optionally about 15%. The width of the tab 333 across the width of the bandage 320 may be between about 5 mm and 300 mm, optionally about 10-30 mm, optionally about 20-25 mm, optionally about 25 mm, optionally about 40-60 mm, optionally about 50 mm, optionally about 65-85 mm, optionally about 75 mm, optionally about 90-110 mm, optionally about 100 mm, optionally about 100-200 mm, optionally about 140-160 mm, optionally about 150 mm. The width of the tab 333 may be between about 20% and 200% of the width of the bandage 320, optionally about 50-150%, optionally about 80-120%, optionally about 100%.

In various embodiments, the dressings 300, 400, 500, 600, 700, 800 and 900 may include any combination of the features described with reference any of the other dressings 300, 400, 500, 600, 700, 800 and 900.

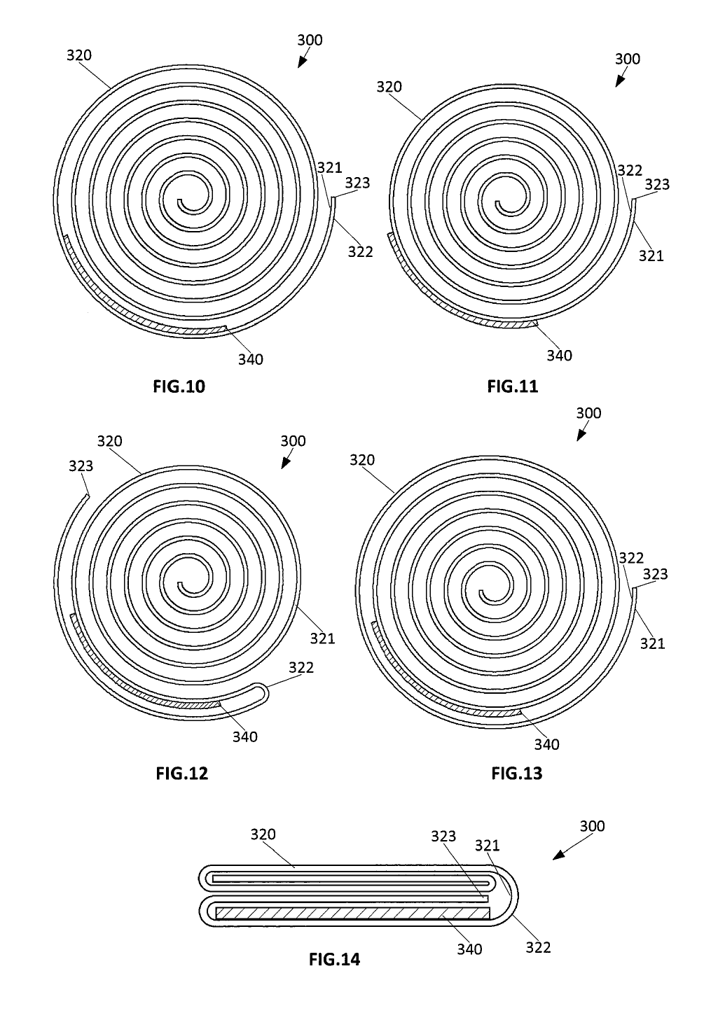

The dressing 300, 400, 500, 600, 700, 800 and 900 may be packaged in different ways for ease of use. In some embodiments, the dressing 300, 400, 500, 600, 700, 800 and 900 may be rolled up or folded, as shown in FIGS. 10 to 14. A second terminal end 329 of the bandage 320 may be rolled first so that the pad 340 ends up nearer the outer portion of the rolled dressing 300, 400, 500, 600, 700, 800 and 900. FIGS. 10 to 14 illustrate different configurations in which the dressing 300, 400, 500, 600, 700, 800 and 900 may be rolled or folded for packaging. The diagrams are schematic illustrations showing a space between windings of the bandage 320; however, in practice, the bandage 320 would be wrapped closely against itself. The bandage 320 may be stretched or left substantially unstretched when packaging, rolling or folding.

The dressing 300, 400, 500, 600, 700, 800 and 900 may be rolled with the inner surface 321 facing towards the centre of the roll, as shown in FIG. 10, so that the pad 340 is held against the roll which reduces the chance of contamination before application. The rolled configuration may be a convenient form for a user to handle and improve ease of use when applying the dressing to a patient. Alternatively, the dressing 300, 400, 500, 600, 700, 800 and 900 may be rolled with the inner surface 321 and pad 340 facing outwards away from the centre of the roll, as shown in FIG. 11. This configuration may be more convenient for unskilled persons who may find it easier to roll the bandage around the appendage while holding the roll against the surface of the appendage.

In the rolled configuration of FIG. 11, the pad 340 is facing outwards and more exposed. In order to better protect the pad 340 from contamination, the terminal end 323 may be folded back over the pad 340 to cohere to the bandage 320 on the other side of the pad 340, as shown in FIG. 12. Alternatively, the terminal end 323 may be rolled further around the rolled bandage 320 in the main rolling direction to wrap around the whole roll and cover the pad 340, as shown in FIG. 13.

Another useful configuration for packaging the dressing 300, 400, 500, 600, 700, 800 and 900 is to fold it, as shown in FIG. 14. Folding the dressing 300, 400, 500, 600, 700, 800 and 900 may allow for a flatter packaging configuration, which may be desirable for certain situations such as when retailing for home use, for example. One folded configuration is for the pad 340 to be left flat, with the terminal end 323 folded over the pad 340, and the rest of the bandage 320 folded over itself in lengths substantially similar to the length of the pad 340 until the entire bandage 320 is folded over to substantially cover the pad 340 and terminal end 323, as shown in FIG. 14. This may allow the bandage 320 to be unfolded easily, as the terminal end 323 will not adhere to the pad 340 and will be easy to unfold from the pad 340.

The dressing 300, 400, 500, 600, 700, 800 and 900 may be enclosed and sealed in any suitable package formed of suitable materials which can maintain a level of sterility of the dressing 300, 400, 500, 600, 700, 800 and 900. Some possible suitable packages are: a blister pack formed of plastic with a paper backing; a blister pack formed of plastic with a foil backing; a rigid plastic container; a sealed envelope formed of paper and/or plastic; and a dressing dispenser. In some embodiments, the packaging may comprise biodegradable plastic. The sealed package containing the dressing 300, 400, 500, 600, 700, 800 and 900 may then be sterilised using gas sterilisation if the package is gas permeable, or irradiation with ionising radiation, for example. Gas sterilisation may comprise gas produced by sublimation of a solid chemical such as iodine, iodine derivatives, naphthalene and naphthalene derivatives. Alternatively, ethylene oxide may be used for gas sterilisation. In some embodiments, the packaging may contain an antimicrobial substance to maintain the sterility of the dressing 300, 400, 500, 600, 700, 800 and 900, such as antimicrobial silver, for example.

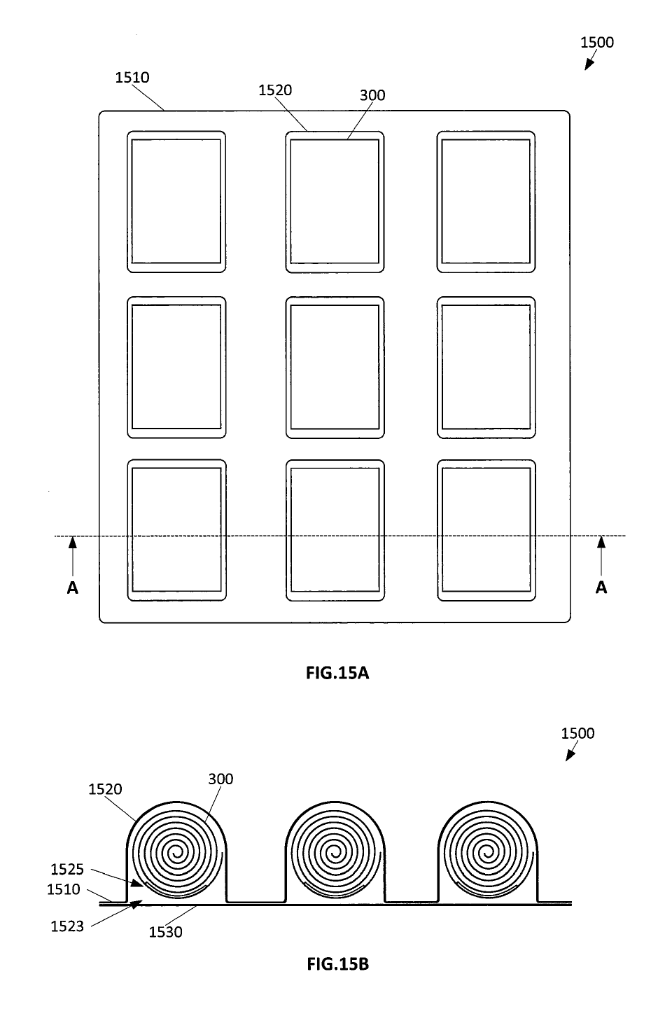

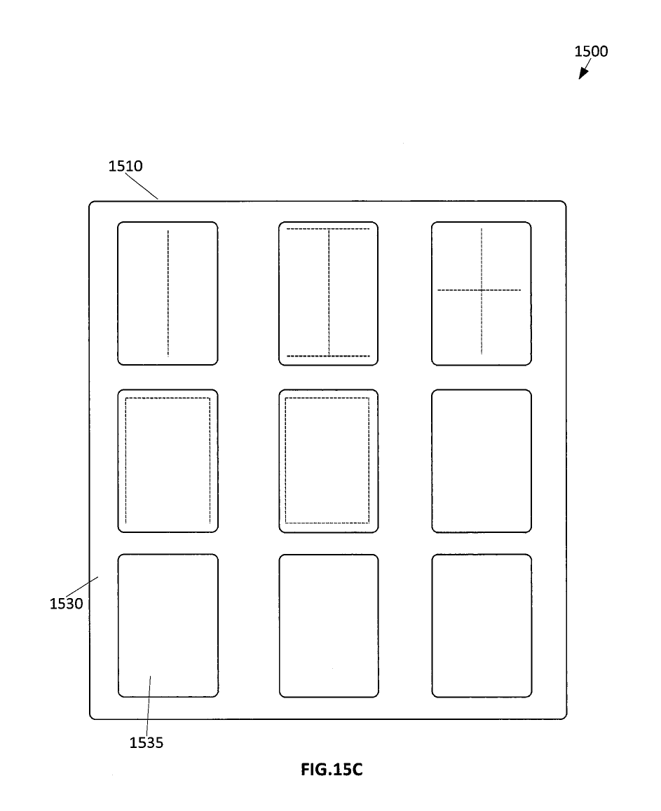

One suitable package for containing the dressing 300, 400, 500, 600, 700, 800 and 900 is a blister pack. The package may comprise a single blister, chamber or compartment containing a single dressing, or the package may comprise multiple chambers, each chamber containing a dressing 300, 400, 500, 600, 700, 800 and 900. An exemplary blister pack 1500 with nine chambers is shown in FIGS. 15A and 15B. The blister pack 1500 comprises a body 1510 defining a substantially flat tray, with chambers 1520 protruding away from the body 1510. Each chamber 1520 defines an opening 1523 and recess 1525 sized to receive a rolled dressing 300, 400, 500, 600, 700, 800 and 900. Once dressings 300, 400, 500, 600, 700, 800 and 900 are placed inside the recesses 1525, the openings 1523 of the chambers 1520 may be covered and sealed with a sheet 1530 having frangible rectangular areas 1535 corresponding to and aligned with the opening 1523 of each chamber 1520. When a dressing 300, 400, 500, 600, 700, 800 and 900 is required, a chamber 1520 may be depressed by a user to push the dressing 300, 400, 500, 600, 700, 800 and 900 against the sheet 1530 to break open one of the rectangular areas 1535 of the sheet 1530 and allow the dressing 300, 400, 500, 600, 700, 800 and 900 to be removed from the package 1500.

The sheet 1530 may include pre-weakened areas in the rectangular areas 1535 such as thinner regions or perforated regions to assist the user to break one of the rectangular areas 1535 of the sheet 1530 and remove a dressing 300, 400, 500, 600, 700, 800 and 900 from a chamber 1520. Various possible perforation patterns are shown in FIG. 15C which may be used in some embodiments. The perforation pattern may comprise a straight centreline extending along a longitudinal axis of the rectangular areas 1535, a centre cross line defined by a straight centreline and a centre cross line extending perpendicularly across the centre of the centreline, or an “I” shape with two straight end cross lines at opposite ends of the rectangular areas 1535 connected by a centreline between them along the longitudinal axis of the rectangular areas 1535. Alternatively, the perforation pattern may extend around part or all of the perimeter of the blister boundary to define a closed rectangle or one edge of the rectangle may be left open to define a “U” shape. The sheet 1530 may be bonded to the body 1510 with adhesive, or by welding or heat sealing. In some embodiments, the sheet 1530 may comprise a gas permeable material such as paper or a synthetic alternative, for example. In other embodiments, the sheet 1530 may comprise a material which is gas impermeable or substantially impermeable to gas, such as foil, metal foil or aluminium foil, for example. The body 1510 and chambers 1520 may comprise any suitable plastics material such as those known for use in medical tablet blister packs. The material properties and thickness may be adapted to provide enough rigidity to the body 1510 and chambers 1520 for them to hold their shape, while still allowing the chambers 1520 to be depressed by a user to eject a dressing 300, 400, 500, 600, 700, 800 and 900 from the pack 1500. In some embodiments, the chambers 1520 may be formed of a transparent plastics material so as to allow visualisation of the dressings 300, 400, 500, 600, 700, 800 and 900 enclosed within the chambers 1520. In some embodiments, the sheet 1530 maybe formed of a transparent plastic. In some embodiments, the blister pack 1500 may comprise biodegradable plastic.

The dressing 300, 400, 500, 600, 700, 800 and 900 may also be packaged in any other suitable package such as a tube or box formed of plastic or cardboard. Different dressings 300, 400, 500, 600, 700, 800 and 900 for different applications may be marked with different indicia such as colour coding, for example. A package for a dressing 300, 400, 500, 600, 700, 800 and 900 may be transparent or include a transparent window so that the dressing size can quickly be identified just by looking at it. Alternatively, or in addition, the size may be written on the outside of the package for a user to read and identify the size and/or type of dressing 300, 400, 500, 600, 700, 800 and 900 within.

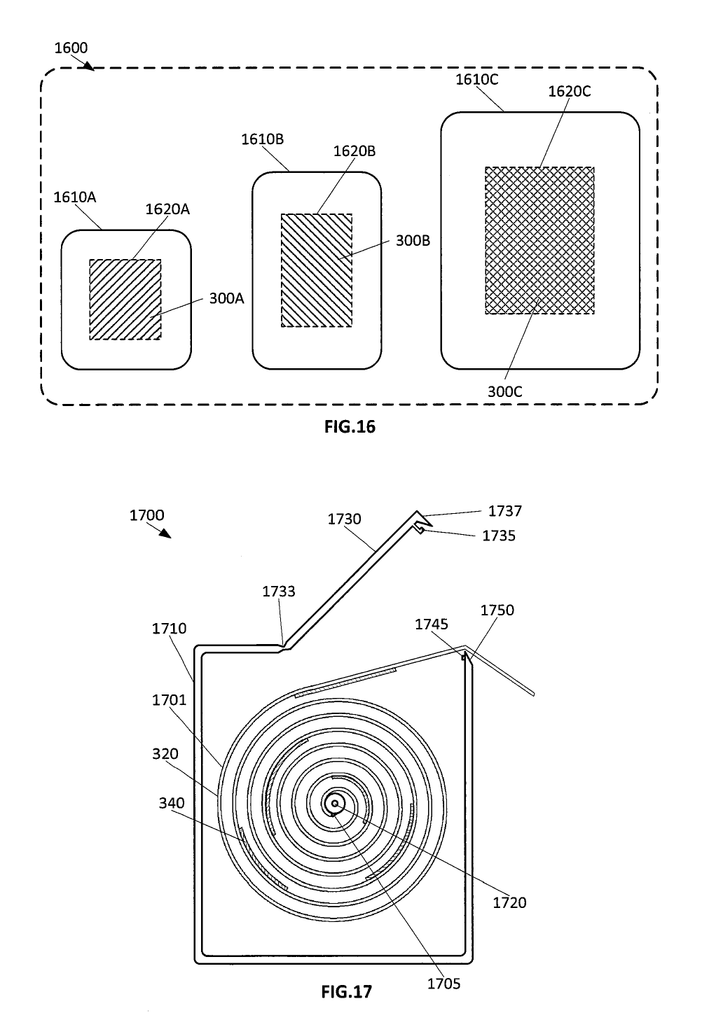

FIG. 16 shows a kit 1600 of different dressings 300, 400, 500, 600, 700, 800 and 900 in different packages 1610. The kit 1600 may comprise a box of the packages 1610. Each package 1610 includes a transparent window 1620 to allow a user to see the dressing 300, 400, 500, 600, 700, 800 and 900 contained within the package 1610. Each dressing 300A, 300B and 300C is coloured differently to readily allow visual differentiation of the differently sizes dressings. In some embodiments, the packages 1610 may themselves be colour coded to indicate the different sizes of the dressings 300, 400, 500, 600, 700, 800 and 900 within. Such a colour coding system, or other set of indicia indicating the different types, sizes and/or absorptive capacities of the dressings 300, 400, 500, 600, 700, 800 and 900 may assist users in quickly identifying and selecting the particular dressing 300, 400, 500, 600, 700, 800 and 900 that they require for a particular application. This may be particularly important in emergency situations in hospitals or for first aid in the field. For example, a particular dressing 300, 400, 500, 600, 700, 800, 900 having a pad 340 with a particular absorptive capacity may be chosen as required for a particular application. The absorptive capacity of the dressing 300, 400, 500, 600, 700, 800, 900 may be printed on the packaging to assist the user in choosing an appropriate dressing 300, 400, 500, 600, 700, 800, 900.

One example colour coding system could comprise 5 different sized dressings 300, 400, 500, 600, 700, 800 and 900 with packages labelled “1”, “2”, “3”, “4”, “5”. The digit on each package may define a transparent area on the packaging that is sized to allow a user to quickly and easily identify the dressing size either by reading the digit or by seeing the colour of the dressing inside. The table below illustrates one possible colour coding system for a particular set of dressing sizes. Alternative sizes could be used according to the ranges described previously and any suitable colour range could be used.

| Dressing | | Absorptive | Pad (mm) | | | Bandage (mm) | | Tail (mm) |
|---|---|---|---|---|---|---|---|---|
| Size | Colour | Capacity (mL) | width | length | thickness | width | length | length |
| 1.0 | Skin tone | 5 | 25 | 50 | 2 | 25 | 250 | 15 |
| 1 | Blue | 5 | 25 | 50 | 2 | 25 | 500 | 30 |
| 2 | Orange | 75 | 50 | 100 | 2 | 50 | 750 | 70 |
| 3 | Green | 300 | 75 | 150 | 2.5 | 75 | 750 | 70 |
| 4 | Yellow | 400 | 100 | 200 | 3 | 100 | 1000 | 70 |
| 5 | White | 500 | 150 | 200 | 4 | 150 | 2000 | 100 |

An alternative packaging arrangement is shown in FIG. 17 in which a number of dressings 300, 400, 500, 600, 700, 800 and 900 connected end to end are rolled up into a roll 1701 and supported in a dispenser 1700 for storing and dispensing the dressings 300, 400, 500, 600, 700, 800 and 900. The dispenser 1700 comprises a body 1710 to contain the dressings 300, 400, 500, 600, 700, 800 and 900, a hub 1720 to support the roll 1701 of dressings 300, 400, 500, 600, 700, 800 and 900, a lid 1730 to open and close the dispenser 1700, and a cutter 1750 for separating individual dressings 300, 400, 500, 600, 700, 800 and 900 from the roll 1701 of dressings 300, 400, 500, 600, 700, 800 and 900. The roll 1701 may be wound onto a hollow central core 1705 and retained to the core 1705 at one end, and the core 1705 of the roll 1701 may be mounted on the hub 1720.

The hub 1720 may be pivotally mounted to the body 1710 to allow the roll of dressings 300, 400, 500, 600, 700, 800 and 900 to be rotated about the hub 1720. The lid 1730 may be connected to the body 1710 by a hinge 1733. The cutter 1750 may comprise a sharp edge, serrated edge or blade. In use, a dressing 300, 400, 500, 600, 700, 800 and 900 may be pulled out of the dispenser, rotating the roll around the hub 1720 as the dressing 300, 400, 500, 600, 700, 800 and 900 is removed. The dressing 300, 400, 500, 600, 700, 800 and 900 may then be separated from the roll by angling it downwards against the cutter 1750, and the edge of the cutter 1750 may assist the user in cutting or tearing the dressing 300, 400, 500, 600, 700, 800 and 900 away from the roll and the dispenser 1700.

The dispenser 1700 may further comprise a protrusion 1745 near the cutter 1750 to engage a latch 1735 near an outer end 1737 of the lid 1730 to keep the lid 1730 closed for storage. The lid 1730 may be opened when a user applies sufficient force to the end 1737 of the lid 1730 to overcome the retaining force between the protrusion 1745 and the latch 1735.

The dressings 300, 400, 500, 600, 700, 800 and 900 on the roll may include pre-weakened areas at the connection points between adjacent dressings 300, 400, 500, 600, 700, 800 and 900. Alternatively, or in addition, the dressings 300, 400, 500, 600, 700, 800 and 900 may include marks printed onto the bandage 320 at the connection points indicating where one dressing 300, 400, 500, 600, 700, 800 and 900 ends and the next begins. This may assist the user to locate the ends of the dressing 300, 400, 500, 600, 700, 800 and 900 and separate the dressing 300, 400, 500, 600, 700, 800 and 900 from the roll and dispenser 1700.

The dispenser 1700 may further comprise a indicator window (not shown) to indicate how many dressings 300, 400, 500, 600, 700, 800 and 900 are left in the dispenser 1700 and alert the user as to when the roll of dressings 300, 400, 500, 600, 700, 800 and 900 needs to be replaced.

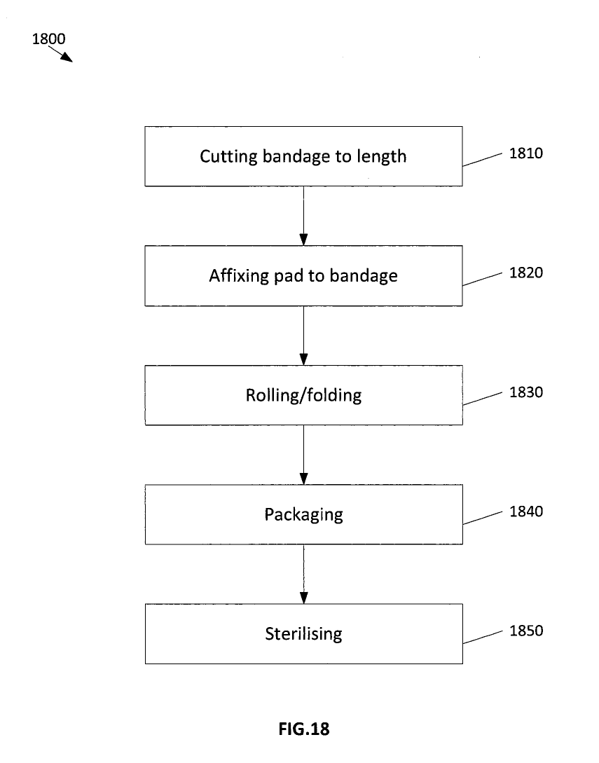

Referring now to FIG. 18, a flow chart is presented showing a method 1800 of manufacturing and packaging the dressings 300, 400, 500, 600, 700, 800 and 900. At 1810, the bandage 320 is cut to length for the particular size that is required. Any suitable length or width of bandage 320 may be chosen for various applications. At 1820, the pad 340 is affixed to the bandage 320 by bonding, stitching, sewing, tacking, gluing, welding or adhering. Various sizes and types of pads 340 may be chosen for different applications. For example, antimicrobial pads may be used, which may be coated or impregnated with silver particles or other treatments to provide a therapeutic benefit, such as decreasing the risk of infection. At 1830, the dressing 300, 400, 500, 600, 700, 800 and 900 may be rolled or folded as desired, as shown in FIGS. 10 to 14, for example. The dressings 300, 400, 500, 600, 700, 800 and 900 may also be arranged on a roll in an interconnected configuration as shown in FIG. 17. At 1840, the dressing 300, 400, 500, 600, 700, 800 and 900 may then be placed and sealed in a package such as a blister pack 1500 or any of the other packages described previously. Any other suitable package may also be used to contain the dressing 300, 400, 500, 600, 700, 800 and 900.

At 1850, the package containing the dressing 300, 400, 500, 600, 700, 800 and 900 may be sterilised. In some cases it may be possible to sterilise the dressing 300, 400, 500, 600, 700, 800 and 900 prior to sealing it in the package; however, sterilisation will generally occur after the dressing 300, 400, 500, 600, 700, 800 and 900 has been sealed in the package. Gas sterilisation may be used if the package is gas permeable. For example, with ethylene oxide. Alternatively, radiation sterilisation may be used to sterilise the dressing 300, 400, 500, 600, 700, 800 and 900 within the package. For example, by irradiating the package and dressing 300, 400, 500, 600, 700, 800 and 900 with ionising radiation.

Although the application has been described in conjunction with specific embodiment thereof, it is evident that many alternatives may and will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present application.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A wound dressing comprising:
a cohesive bandage having a length and a width, the length being substantially greater than the width; and
an absorbent pad affixed to the bandage near to but spaced from a terminal end of the bandage so as to form a tail portion between the pad and the terminal end of the bandage.

2. A dressing according to claim 1, wherein the length of the tail portion is between about 10 mm and about 100 mm.

3. A dressing according to claim 1, wherein the length of the tail portion is between about 20 mm and about 40 mm.

4. A dressing according to claim 1, wherein the length of the tail portion is between about 25 mm and about 35 mm.

5. A dressing according to claim 1, wherein the length of the tail portion is between about 60 mm and about 80 mm.

6. A dressing according to claim 1, wherein the length of the tail portion is between about 65 mm and about 75 mm.

7. A dressing according to claim 1, wherein the length of the tail portion is between about 5% and 10% of the length of the bandage.

8. A dressing according to claim 1, wherein the length of the tail portion is about 6-8% of the length of the bandage.

9. A dressing according to any one of claims 1 to 8, wherein the pad comprises at least one therapeutic additive.

10. A dressing according to claim 9, wherein the at least one additive is selected from the group of classes comprising: medication, drugs, ointments, antimicrobials, antibiotics, antiseptics, coagulants, hydrogels, analgesics, anaesthetics, anti-inflammatories and antihistamines.

11. A dressing according to claim 9 or 10, wherein the at least one additive is selected from the group comprising: silver derivatives, iodine derivatives, petroleum derivatives, saline, chitosan, hydrofibres, hydrogels, alginate, Melaleuca oil, sphagnum moss or substances derived therefrom, antimicrobial oils and honey.

12. A dressing according to any one of claims 9 to 11, wherein the at least one additive comprises a coagulant selected from the group comprising: cellulose-based agents, gelatin-based agents, platelet rich plasma, collagen-based agents, fibrin-based agents, thrombin-based agents, chitin-based agents, chitosan-based agents and mineral-based agents.

13. A dressing according to any one of claims 9 to 12, wherein the at least one additive comprises a hydrogel including Melaleuca oil.

14. A dressing according to any one of claims 1 to 13 further comprising a cold pack.

15. A dressing according to claim 14, wherein the cold pack can be activated to start an endothermic reaction to provide a cooling effect.

16. A dressing according to claim 14 or 15 further comprising one of a sleeve, slot, pocket or pouch to receive the cold pack.

17. A dressing according to any one of claims 14 to 16 wherein the cold pack is disposed between the bandage and the pad.

18. A dressing according to any one of claims 1 to 17 further comprising a non-adhesive tab affixed to the terminal end of the bandage, the tab being formed of a different material than a material of the bandage.

19. A dressing according to any one of claims 1 to 18, wherein the width of the pad in a direction across the width of the bandage is between about 10 mm and about 30 mm.

20. A dressing according to any one of claims 1 to 18, wherein the width of the pad in a direction across the width of the bandage is between about 40 mm and about 60 mm.

21. A dressing according to any one of claims 1 to 18, wherein the width of the pad in a direction across the width of the bandage is between about 65 mm and about 85 mm.

22. A dressing according to any one of claims 1 to 18, wherein the width of the pad in a direction across the width of the bandage is between about 90 mm and about 110 mm.

23. A dressing according to any one of claims 1 to 18, wherein the width of the pad in a direction across the width of the bandage is between about 140 mm and about 160 mm.

24. A dressing according to any one of claims 1 to 23, wherein the width of the pad is between about 25% and 400% of the length of the pad.

25. A dressing according claim 24, wherein the width of the pad is between about 40% and 100% of the length of the pad.

26. A dressing according to any one of claims 1 to 25, wherein the width of the pad is between about 50% and about 200% of the width of the bandage.

27. A dressing according to claim 26, wherein the width of the pad is between about 75% and about 120% of the width of the bandage.

28. A dressing according to claim 27, wherein the width of the pad is approximately equal to the width of the bandage.

29. A dressing according to any one of claims 1 to 28, wherein the dressing is sized to wrap around a human digit.

30. A dressing according to any one of claims 1 to 28, wherein the dressing is sized to wrap around a human limb or human head.

31. A dressing according to any one of claims 1 to 28, wherein the dressing is sized to wrap around a human torso.

32. A dressing according to any one of claims 1 to 31, wherein the pad comprises one or more foldable wings to extend a contact area of the pad.

33. A dressing according to any one of claims 1 to 32, wherein the dressing is a compression dressing adapted to exert a compressive force to a body part when wrapped around the body part.

34. A compression dressing, comprising:
an elastic bandage strip being designed for exerting a compressive force when wrapped around a body part sufficient to hold the compression dressing in place; and
an absorbent pad being affixed to an inner side of said elastic bandage strip and close to a terminal end of said elastic bandage strip.

35. The compression dressing of claim 34, wherein said elastic bandage strip has self-adhesive properties and is designed not to adhere to clothing, hair, skin or latex gloves.

36. The compression dressing of claim 34, wherein said elastic bandage strip is constructed so as to have self-adhesive properties sufficient to cause two adjacent layers of the compression dressing to remain adhered to one another under elastic extension or pressure.

37. The compression dressing of claim 34, wherein said elastic bandage strip is constructed of non-woven elastomeric fibres or of woven cotton type having firm elastic extendibility in longitudinal and vertical directions, and an adhesive attached thereto, and wherein said elastic bandage strip has self-adhesive properties sufficient to cause two adjacent layers of the compression dressing to remain adhered to one another without the use of a fastening mechanism.

38. The compression dressing of claim 34, wherein said absorbent pad is attached to the elastic bandage strip by stitching, welding, needle tacking or bonding.

39. The compression dressing of claim 34, wherein said absorbent pad is constructed of foamed, woven or nonwoven material of natural or synthetic fibres.

40. The compression dressing of claim 34, wherein the compression dressing is loosely rolled into a roll and packaged in a sterile packaging.

41. The compression dressing of claim 34, wherein said absorbent pad has a width equal to or smaller than that of said elastic bandage strip.

42. The compression dressing of claim 34, wherein a distance between said absorbent pad and said terminal end of said elastic bandage strip is from 10 mm to 100 mm.

43. The compression dressing of claim 42, wherein the distance between said absorbent pad and said terminal end of said elastic bandage strip is from 50 mm to 70 mm.

44. The compression dressing of claim 34, wherein a non-adhesive covering is arranged on the exposed surface of said absorbent pad.

45. The compression dressing of claim 34, wherein said elastic bandage strip is a cohesive bandage.

46. A wound dressing product comprising a package and a dressing according to any one of claims 1 to 45 sealed within the package.

47. A wound dressing product according to claim 46, wherein at least part of the package is formed of a material which allows the dressing to be sterilised within the package.

48. A wound dressing product according to claim 47, wherein the material has a gas permeability high enough to allow gas sterilisation of the dressing within the package.

49. A wound dressing product according to any one of claims 46 to 48, wherein at least part of the package is formed of a transparent material to allow a user to see at least part of the dressing while the dressing remains sealed within the package.

50. A wound dressing product according to any one of claims 46 to 49, wherein the dressing is in a rolled or folded configuration within the package.

51. A wound dressing product according to any one of claims 46 to 50, wherein the package comprises a blister pack including one or more chambers, each chamber defining a recess and an opening, each recess containing one of the dressings, and each opening sealed by a sheet.

52. A wound dressing product according to any one of claims 46 to 49, wherein the package comprises a dressing dispenser containing a roll of a plurality of the dressings connected end to end, and wherein the dressings are manually separable from one another.

53. A wound dressing product according to claim 52, wherein the roll of dressings further comprises indicia printed on the dressings to indicate where one dressing ends and the next adjacent dressing begins.

54. A wound dressing product according to claim 52 or 53, wherein the dispenser further comprises an edge or blade for separating dressings from the roll.

55. A wound dressing kit comprising a plurality of wound dressing products according to any one of claims 46 to 54.

56. A wound dressing kit according to claim 55 further comprising a kit package to contain the plurality of wound dressing products.

57. A wound dressing kit according to claim 55 or 56, wherein the plurality of products includes a range of different dressing sizes.

58. A wound dressing kit according to any one of claims 55 to 57, wherein the plurality of products includes a range of different dressing types.

59. A wound dressing kit according to any one of claims 55 to 58, wherein each package of the plurality of products includes indicia to indicate at least one of a size and a type of the dressing contained within.

60. A wound dressing kit according to any one of claims 55 to 59, wherein the plurality of products are colour coded by at least one of a size and a type such that each dressing of the plurality of products is coloured to indicate a respective at least one of size and type of the dressing.

61. A method of manufacturing a wound dressing product according to any one of claims 46 to 54, the method comprising: forming the dressing; enclosing the dressing in the package;
and sealing the package.

62. A method according to claim 61 further comprising sterilising the dressing in the package.

63. A method of manufacturing a dressing comprising:
forming a cohesive bandage having a length and a width, the length being substantially greater than the width; and
affixing an absorbent pad to the bandage near to but spaced from a terminal end of the bandage so as to form a tail portion between the pad and the terminal end of the bandage.

64. A method according to claim 63 further comprising providing the pad with one or more therapeutic additives.

65. A method according to claim 63 or 64 further comprising sealing the dressing in a package.

66. A method according to claim 65 further comprising sterilising the dressing within the sealed package.
